(19) United States
(12) Patent Application Publication
(10) Pub. No.: US 2005/0096729 A1
(43) Pub. Date: May 5, 2005

(54) Methods and apparatus for intraluminal device

(76) Inventor: James V. Donadio III, Victoria, MN (US)

Correspondence Address: Law Offices of Daniel J. Noblitt, LLC;Attn: Daniel J. Noblitt, 3370 North Hayden Road, Suite 123, Box 258, Scottsdale, AR, 85251, US

(21) Appl. No.: 10/698,692

(22) Filed: Oct. 31, 2003

(51) Int. Cl. A61F 2/06

(52) U.S. Cl. 623/1.15; 264/317; 264/81; 249/61

(57) ABSTRACT

According to a preferred embodiment of the present invention, a method and apparatus for forming a device, such as a stent or catheter, includes a device-forming element comprising a mandrel having a device pattern defined therein, and the device in the device pattern. The device is separated from the mandrel by dissolving the mandrel.

### FIELD OF THE INVENTION

The invention relates to methods and apparatus for small devices.

### BACKGROUND OF THE INVENTION

Small devices are widely used in a variety of applications, particularly in medical applications for placement in a lumen of a patient. For example, intraluminal devices such as stents are commonly used to treat obstructed coronary arteries. Typically, such stents are reticulated tubular structures. The stents are placed on a balloon tip catheter and advanced through the patient's blood vessels to an occluded artery. At the occluded site, the balloon is expanded to enlarge the stent's diameter. With the stent so enlarged, the balloon is deflated and the catheter is removed from the patient leaving the enlarged stent in place with the intent that the formerly occluded site is held open by the stent.

In addition to advancing stents as described above, catheters are used in a wide variety of applications. Accordingly, catheters are available in a wide variety of designs. Many such designs require extremely small diameter and flexible catheters. For example, in neurological applications, catheters must be extremely narrow and flexible in order to be advanced through the patient's vasculature to a desired site.

To achieve the small diameters and desired flexibility as well as other properties, stents, catheters and other tubular intraluminal devices may include hollow tubes fabricated with a plurality of openings formed through the walls of the tube. Because intraluminal devices have such small diameters, it is often difficult to fabricate them. For example, stents may be formed by laser-machining a solid-walled metal tube. Through accurate control of the laser, the laser is axially and circumferentially moved relative to the stent and selectively energized to form holes through the wall of the tube and form the stent. Laser machining, however, is very costly and has limited effectiveness.

Other processes form reticulated intraluminal devices like stents and catheters without requiring the use of lasers and their disadvantages. In one such process, a chemical resistant coating is applied to a tube. Using a photo-mask, portions of the coating are exposed to a light source. The exposed portions are removed in a developing process to expose a pattern on the surface of the tube. The tube is then chemically etched to remove tube material exposed through the developed pattern. Following such etching, the remainder of the coating is removed.

Chemical etching, however, presents certain challenges. For example, when a chemical etchant is applied to a limited exposed area on the exterior of a tube, the etchant does not dissolve perfectly radially toward the center of the tube. This tendency precludes certain stent pattern geometries which can be formed in a chemical etching process. For example, intricate, narrow corners are difficult to form in stents and catheters made by chemical etching. Further, the non-radial etching path can result in the formation of stent or catheter walls being non-radial relative to the tube's axis. These non-radial walls intersect with the interior cylindrical surface of the stent tube in such a manner that sharp edges can be formed.

### SUMMARY OF THE INVENTION

According to a preferred embodiment of the present invention, a method and apparatus for forming a device, such as a stent or catheter, includes a device-forming element comprising a mandrel having a device pattern defined therein, and the device in the device pattern. The device is separated from the mandrel by dissolving the mandrel.

### BRIEF DESCRIPTION OF THE DRAWINGS

A more complete understanding of the present invention may be derived by referring to the detailed description when considered in connection with the following illustrative figures. In the following figures, like reference numbers refer to similar elements and steps.

FIGS. 1A-B are a side-elevation view and a cross-section view taken along line 1-1 of a device-forming element according to various aspects of the present invention;

FIGS. 2A-B are a side-elevation view and a cross-section view taken along line 2-2 of an etched mandrel with undeveloped photoresist stripped away;

FIGS. 3A-B are a side-elevation view and a cross-section view taken along line 3-3 of a finished intraluminal device fabricated following removal of the mandrel material from the intraluminal device;

FIGS. 4A-B are a side-elevation view and a cross-section view taken along line 4-4 of the mandrel coated with a photoresist;

FIGS. 5A-B are a side-elevation view and a cross-section view taken along line 5-5 of the photoresist-coated mandrel following light imaging for imaging a desired device pattern onto the photoresist;

FIGS. 6A-B are a side-elevation view and a cross-section view taken along line 6-6 of the imaged mandrel with the imaged pattern developed and removed;

FIGS. 7A-B are a side-elevation view and a cross-section view taken along line 7-7 of the developed mandrel following etching of the portions of the mandrel exposed through the developed pattern; and

FIGS. 8A-B are a side-elevation view and a cross-section view taken along line 8-8 of the stripped mandrel of FIG. 11 following deposit of a stent material.

Elements and steps in the figures are illustrated for simplicity and clarity and have not necessarily been rendered according to any particular sequence. For example, steps that may be performed concurrently or in different order are illustrated in the figures to help to improve understanding of embodiments of the present invention.

### DETAILED DESCRIPTION OF EXEMPLARY EMBODIMENTS

The present invention is described partly in terms of functional components and various processing steps. Such functional components may be realized by any number of components configured to perform the specified functions and achieve the various results. For example, the present invention may employ various elements, materials, intraluminal devices, molds, device materials, device patterns, and the like, which may carry out a variety of functions. In addition, the present invention may be practiced in conjunction with any number of applications, environments, conduits, and/or intraluminal devices, and the systems described are merely exemplary applications for the invention. Further, the present invention may employ any number of conventional techniques for manufacturing, assembling, molding, dissolution, and the like.

A device preparation system for generating a small device according to various aspects of the present invention may be implemented in conjunction with any desired device, such as a small but intricate component. In the present embodiment, the device preparation system is implemented to create an intraluminal device for placement within a conduit, such as a stent, catheter, or other device for deployment within a blood vessel or other small conduit. Generally, the present device preparation system is configured to provide a stent having selected characteristics. The device preparation system suitably operates in conjunction with a mandrel having a device pattern that can be separated from the intraluminal device.

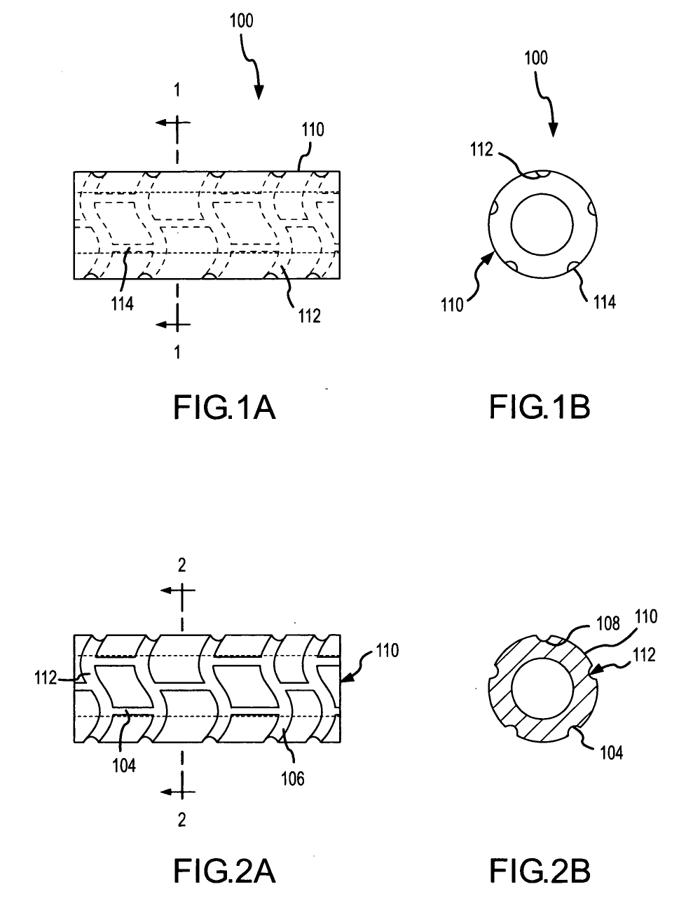

For example, referring to FIGS. 1A-B, a device preparation system 100 according to various aspects of the present invention comprises a mandrel 110 having a device pattern 112 defined in a surface of the mandrel 110. In the present embodiment, the device preparation system 100 is a stent-forming element configured to generate a stent 114 having selected characteristics, though the device preparation system may be configured in any suitable manner to generate a particular device. The mandrel 110 may comprise any suitable element for defining the device pattern 112. The mandrel 110 may be configured to support the device pattern 112 having a particular configuration, such as a rod or tube for a vascular stent or catheter. The mandrel 110 may be otherwise shaped, however, for any desired device pattern 112.

The mandrel 110 is formed of a substantially rigid material, such as stainless steel, molybdenum, or tungsten. The mandrel 110 material is also suitably deformable, such as being subject to melting, softening, or dissolution, in a specific environment, such as when exposed to heat, moisture, selected chemicals, or the like. For example, the mandrel 110 may be comprised of a steel, alloy, or ceramic, such as CATAMOLD M2 brand tool steel provided by BASF, which is a substantially rigid material at room temperature and while exposed to ordinary atmosphere. The material may be dissolved when exposed to selected chemicals, such as nitric acid, sulfuric acid, and/or hydrochloric acid.

Referring now to FIGS. 2A-B, in the present embodiment, the mandrel 110 is a hollow tube or solid rod having a thickness sufficient to contain the device pattern 112, such as a depth of about 0.010 inch or about 0.25 mm. The present mandrel 110 is at least as long as the desired length of the stent 114 and has an outside diameter approximately equal to the outside diameter of the stent 114, such as about 0.050 inch (about 1.25 mm). Such dimensions, however, are provided solely to demonstrate a possible mandrel. The outside diameter of the mandrel 110 may, however, be greater than the outside diameter of the stent 114.

The mandrel 110 includes a surface in which the device pattern 112 is defined. In the present embodiment, the device pattern 112 is defined in the outer surface of the mandrel 110, though any suitable surface may define the device pattern 112. The present device pattern 112 comprises one or more grooves, ridges, cavities, bumps, or other topographical characteristics formed in or on the relevant surface of the mandrel 110. The device pattern 112 may include channels or cavities having different widths, depths, and lengths, as well as other characteristics, such as rounded or flat walls and/or bottom surfaces. For example, the present device pattern 112 has axial and circumferential portions 104, 106 corresponding to and substantially matching the desired device pattern 112. All or a portion of the device pattern 112 may also include a concave cross-section such that the device pattern 112 has a rounded trough surface at the bottom 108, which tends to impart a rounded surface to the device.

The device may comprise any suitable device to be made in conjunction with the mandrel 110. In the present embodiment, the device comprises an intraluminal device for deployment within a conduit, particularly a small conduit such as a blood vessel or a biliary duct. The intraluminal device is formed in the device pattern 112 and is suitably separated from the mandrel 110 for deployment. The device may be comprised of any suitable material for the particular application, environment, and fabrication techniques.

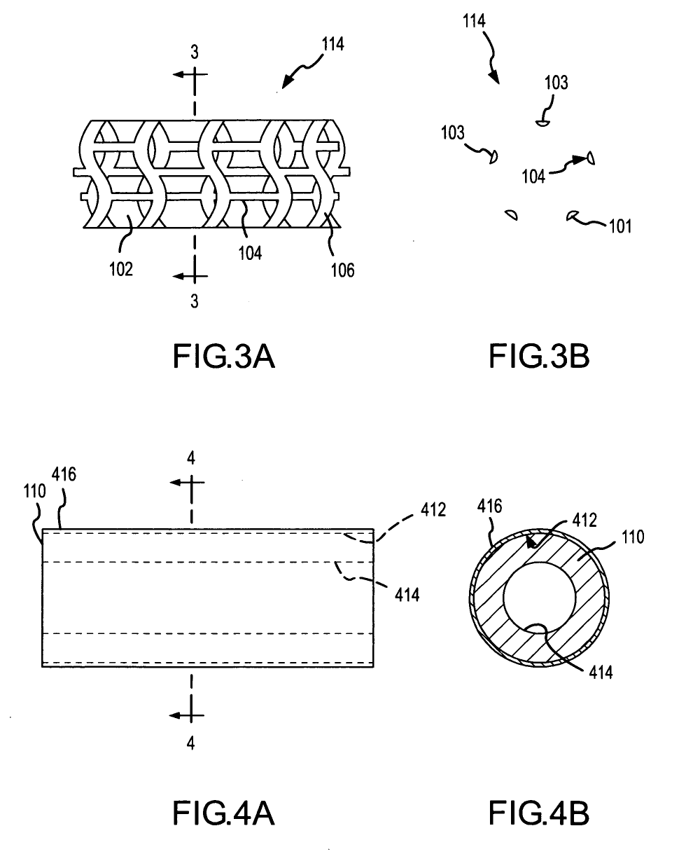

The intraluminal device may be configured in any suitable manner for a selected purpose. Referring to FIGS. 3A-B, in the present embodiment, the intraluminal device comprises the stent 114 configured for any suitable application or environment, such as to keep a formerly blocked passageway open, relieve pressure on a blood vessel, or deliver drugs. The intraluminal device may comprise, however, any suitable device, such as a catheter, acoustic transducer housing, optical instrument housing, or other device for deployment in the relevant conduit.

In the present exemplary embodiment, the stent 114 comprises a tube of about 0.050 inch (about 1.25 mm) outside diameter, 0.75 inch (about 19 mm) length, and 0.005 inch (about 0.13 mm) wall thickness. Such dimensions, however, are provided solely to demonstrate a possible intraluminal device. The stent 114 may include any appropriate structure to facilitate delivery and deployment of the stent 114. For example, the present stent 114 suitably includes a reticulated structure with a plurality of openings 102 defined by axial and circumferential portions 104, 106. The stent 114 may be comprised of any appropriate material, such as a relatively durable and biocompatible material, for example titanium, tantalum, niobium, zirconium, platinum, stainless steel, or a polymer.

The device may be configured in any suitable configuration for a desired application or environment. The device may include one or more openings, arms, joints, appendages, and the like having selected thicknesses, shapes, radii, lengths, or other features to provide desired characteristics, such as stiffness and/or durability in one or more directions, rounded edges at selected locations, or selected rough surfaces for engaging coatings or other materials. For example, the stent 114 may include portions having rounded surfaces, tending to avoid sharp edges on the interior surface 101 of the stent 114 that might interfere with deployment using a balloon catheter or disrupt blood flow. The outer surface 103 of the stent 114 is suitably cylindrical or otherwise configured to conform to the interior surface of the lumen. In addition, the stent 114 may include structural members of varying thicknesses and dimensions. The stent 114 may also include multiple layers of materials. For example, a radiopaque layer may be disposed between two layers of other materials, such as materials selected for better tissue or blood compatibility. The stent 114 may also include a porous material, which may be saturated with a fluid such as a drug.

The device preparation system 100 may be implemented in any suitable manner to create the mandrel 110 and/or the device. The mandrel 110 and the device pattern 112, for example, may be formed by chemical milling, laser milling, electro-discharge machining (EDM), casting, molding, and/or machining. Any suitable process or technique may be used to generate the mandrel 110 and form the device pattern 112 in the relevant surface of the mandrel 110. The particular process or technique utilized may be selected according to any appropriate criteria, such as cost, precision requirements, compatibility with the materials, or other relevant criteria.

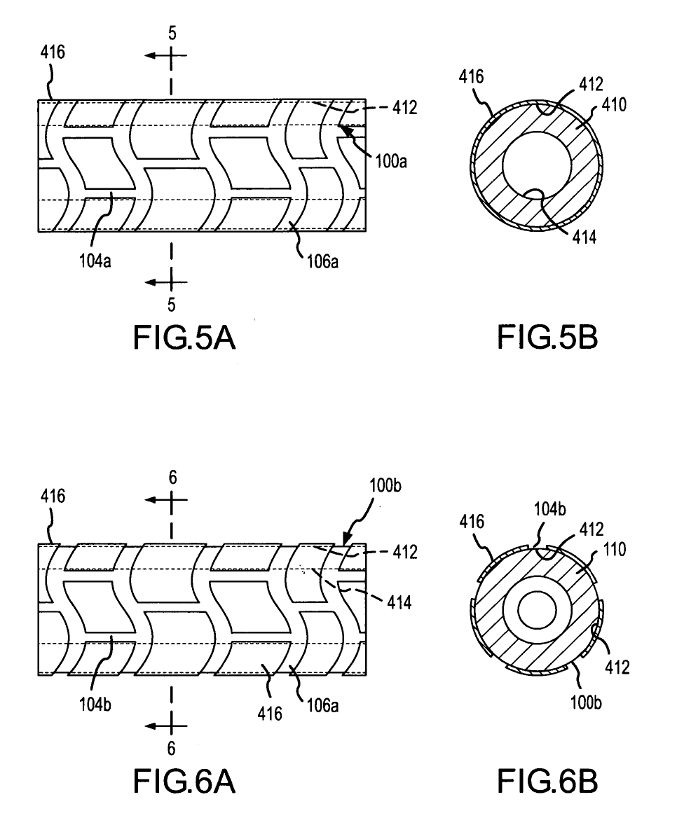

For example, in one embodiment, the device pattern 112 may be formed in the outer surface of the mandrel 110 using a photo-etching process. Referring to FIGS. 4A-B, a photo-resist coating 416, such as a coating about 0.0003 inch, or about 0.008 mm, thick) may be placed on the outer cylindrical surface 412 of the mandrel 110. The coating 416 is resistant to chemical etching. Referring to FIGS. 5A-B, the coating 416 is exposed to a light source through a photo-mask having a light transparent or opaque pattern corresponding to the desired device pattern 112, which photo-sensitizes a pattern 100a on the coating 416. As an alternative, the pattern 100a could be laser printed. The photo-sensitized pattern 100a has axial and circumferential portions 104a, 106a corresponding to and matching the desired device pattern 112.

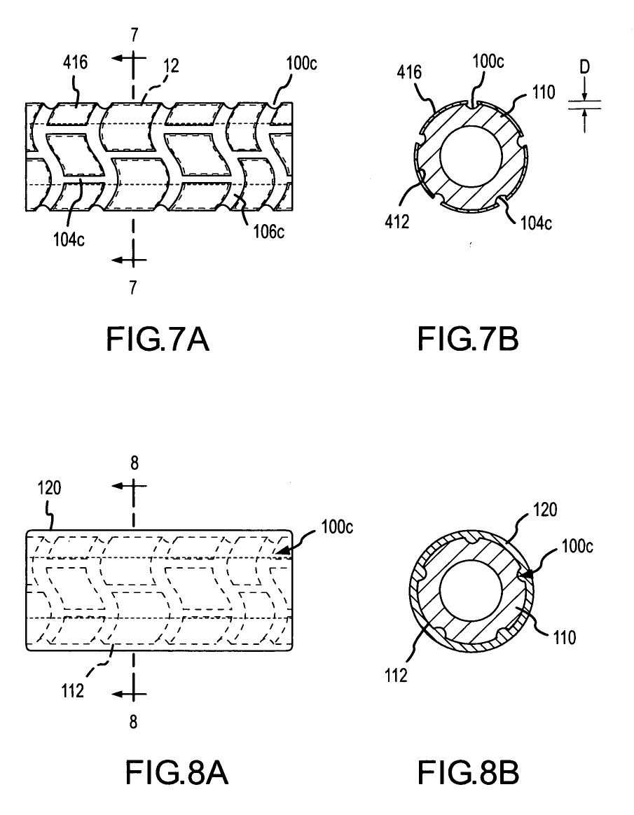

The sensitized pattern 100a is then suitably removed in a developing process. Referring to FIGS. 6A-B, the developing process exposes a pattern 100b on the surface 12 of the mandrel 110. The exposed surface pattern 100b has axial and circumferential portions 104b, 106b corresponding to and matching the desired device pattern 112. With a surface pattern 100b on the mandrel 110 so exposed and with the remainder of the mandrel surface 12 protected by the undeveloped coating 16, the mandrel 110 is suitably etched with an etching solution to form the device pattern 100c. The etching process is suitably discontinued before the etchant dissolves through the thickness of the mandrel 110. For example, referring to FIGS. 7A-B, the etching process may controlled to terminate the etching after the etching has penetrated a desired depth D into the mandrel 110 without penetrating through the interior surface 14 of the mandrel 110. The desired depth D may be selected to approximate the desired wall thickness of the stent 114. Alternatively, the device pattern 112 may be formed to a greater depth, and excess thickness may be removed later, such as through a grinding or other metal removing process. After the device pattern 100c is etched into the mandrel 110, the remaining, unexposed area of coating 16 is removed.

In an alternative embodiment, the mandrel 110 having the device pattern 112 may be formed by molding, for example by powder injection molding. For example, a mold may be prepared, such as a four-cavity mold, having an inverse of the device pattern 112 formed in the walls of the mold. A suitable metal material, such as a fine metal powder mixed with an appropriate plastic binder, may be injected into the mold at a selected pressure. The mandrel 110 of the present embodiment is comprised of a powder injection molding material, such as a metal or ceramic injection molding material, like a metal injection molding tool steel such as BASF Catamold M2, which is a substantially rigid material at room temperature and while exposed to ordinary atmosphere.

After molding, the molded mandrel material is debound, for example by heating, a solvent, or other technique, and sintered at a selected temperature to fuse the fine powdered particles into a solid shape that substantially retains all of the mold's features, including the device pattern 112 on the exterior surface of the mandrel 110. In the present embodiment, the material is suitably debound by exposing the mandrel 110 to selected chemicals and/or other environment, such as heat and/or a catalyst. During this process, the molded mandrel 110 may shrink. The resulting mandrel 110 may then be finally processed, for example to polish surfaces, remove burrs, and the like.

The intraluminal device may be formed on the mandrel 110 in any appropriate manner. A stent material 120 may be deposited into the device pattern 112 to assume the desired device pattern 112. For example, referring to FIGS. 8A-B, the stent material 120 may be vapor deposited onto the mandrel 110 in a titanium layer 20. The stent material 120 fills the device pattern 112 and coats the remaining outer surface 12 of the mandrel 110. Alternatively, the stent material 120 may be deposited on the mandrel 110 by casting the stent material 120 as a molten material poured into the depressed area and retained by an outer casing, electro-forming, forging or crimping (such as by placing an outer tube of stent material 120 around the mandrel 110 and forcibly urging the stent material 120 into the device pattern 112), sputter deposition, ion plating, or other suitable method. The stent material 120 may be deposited to overfill the device pattern 112 such that excess stent material 120 is deposited on the outer surface of the mandrel 110 and above the device pattern 112.

The intraluminal device may also be configured with multiple layers of materials to achieve desired characteristics. For example, a first material may be deposited using a first method, and then the same or a different material may be deposited over or in between the first material. The process may be repeated to generate multiple layers having desired characteristics. For example, using different layers may provide selected stiffness characteristics in different directions, include a radiopaque material between two more biocompatible materials, add porous layers for elution of drugs, or provide other desired features and characteristics.

The stent material 120 may also include porous materials. The porous materials may be configured in any suitable manner and for any suitable purpose, such as for absorbing drugs. The porous material may be any suitable material, and the intraluminal device may be comprised fully or partially of the porous material. For example, the intraluminal device may include an outer layer of porous material deposited on a solid layer of device material.

In the present embodiment, the porous material is deposited by depositing a mixture of two materials and then removing one of them. For example, an alloy of gold and silver, such as an approximately 76% silver and 24% gold alloy, may be deposited substantially homogeneously on the mandrel 110 or on an outer surface of a layer of the intraluminal device. The deposited alloy may then be exposed to a solvent, such as an acid, to dissolve the silver. In addition, an electrochemical potential may be applied to the alloy to promote dissolution of the silver. When the silver is substantially dissolved, the remaining gold structure is porous.

After the stent material 120 has been placed on the mandrel 110, the stent-forming element comprising the mandrel 110 and the intraluminal device may be processed to achieve desired characteristics for the intraluminal device. For example, referring again to FIGS. 1A-B, excess stent material 120 may be removed from the stent-forming element, such as by grinding the outer surface of the stent-forming element until the mandrel 110 material is exposed. As a result, the only stent material 120 on the mandrel 110 is the stent material 120 in the device pattern 112. The stent material 120 includes axial and circumferential portions 104, 106 filling and conforming to the axial and circumferential portions 104c, 106c of the device pattern 112.

In addition, the exposed surface of the device may be treated in any appropriate manner to provide features or characteristics to the intraluminal device. For example, the outer surface of the device may be treated with a coating, for example to provide a porous or radiopaque outer surface. In addition, the intraluminal device may be treated by mechanical modification. Because the intraluminal device remains supported the mandrel 110, the intraluminal device may be mechanically treated, such as cut, ground, or shaped, with less susceptibility to unintended deformation or degradation. Further, if the intraluminal device includes multiple layers, the outer layer may be treated before separation of the intraluminal device from the mandrel 110. For example, if the outer layer is to be treated to create porosity, the intraluminal device may be treated while on the mandrel 110. The outer surface may also receive any additional treatment, such as exposure to a drug or other chemical to impregnate the intraluminal device.

The mandrel 110 may also be adjusted, for example to expose additional area on the intraluminal device for additional treatment. For example, the mandrel 110 may be partially etched such that an outer portion of the mandrel 110 is removed while the intraluminal device remains in position. The exposed area of the intraluminal device may then be treated, for example with a coating or mechanical treatment.

When formation and treatment of the intraluminal device while on the mandrel 110 is completed, the intraluminal device may separated from the mandrel 110. The intraluminal device may be removed from the mandrel 110 in any suitable manner. For example, in the present embodiment, the mandrel 110 may be dissolved in a solvent that dissolves the mandrel 110 but not the stent material 120. When the mandrel 110 is dissolved sufficiently, the intraluminal device separates from the mandrel 110. The intraluminal device may then be finally processed and prepared for delivery and deployment. For example, the intraluminal device may be smoothed to remove burrs or other unwanted elements. In addition, the intraluminal device may be saturated with a fluid, such as a drug. The intraluminal device may then be readied for delivery and deployment.

The particular implementations shown and described are illustrative of the invention and its best mode and are not intended to otherwise limit the scope of the present invention in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The present invention has been described above with reference to a preferred embodiment. However, changes and modifications may be made to the preferred embodiment without departing from the scope of the present invention. These and other changes or modifications are intended to be included within the scope of the present invention.

1. A method of making a stent, comprising:
providing a mandrel having a stent pattern;
filling the stent pattern with a stent material; and
dissolving the mandrel.

2. A method of making a stent according to claim 1, further comprising providing a mandrel mold having the stent pattern, and wherein providing the mandrel includes forming the mandrel in the mandrel mold.

3. A method of making a stent according to claim 2, wherein forming the mandrel in the mandrel mold includes metal injection molding the mandrel in the mandrel mold.

4. A method of making a stent according to claim 2, wherein forming the mandrel includes placing powdered metal in the mandrel mold.

5. A method of making a stent according to claim 1, wherein the stent pattern includes a rounded trough surface.

6. A method of making a stent according to claim 1, wherein filling the stent pattern with the stent material includes depositing the stent material in the stent pattern using chemical vapor deposition.

7. A method of making a stent according to claim 1, wherein filling the stent pattern with the stent material includes filling the stent pattern with a porous stent material.

8. A method of making a stent according to claim 7, further comprising saturating the porous stent material with a fluid.

9. A method of making a stent according to claim 1, further comprising forming pores in at least a portion of the stent material.

10. A method of making a stent according to claim 9, further comprising saturating the porous portion of the stent material with a fluid.

11. A method of making a stent according to claim 1, wherein filling the stent pattern with the stent material includes:
providing a first layer of a first stent material in the stent pattern; and
providing a second layer of a second stent material in the stent pattern.

12. A method of making a stent according to claim 11, wherein the second layer includes a radiopaque material.

13. A method of making a stent according to claim 1, wherein filling the stent pattern comprises overfilling the stent pattern.

14. A method of making a stent according to claim 13, further comprising removing an excess portion of the stent material from the mandrel.

15. A method of making a stent according to claim 1, wherein dissolving the mandrel comprises partially dissolving the mandrel and then fully dissolving the mandrel, and wherein the method further comprises changing the configuration of the stent material after the partial dissolution of the mandrel and before the full dissolution of the mandrel.

16. A stent formed by the process of:
forming a mandrel having a stent pattern;
filling the stent pattern with a stent material; and
dissolving the mandrel.

17. A stent formed by the process of claim 16, wherein the process further comprises providing a mandrel mold having the stent pattern, and wherein providing the mandrel includes forming the mandrel in the mandrel mold.

18. A stent formed by the process of claim 17, wherein forming the mandrel in the mandrel mold includes metal injection molding the mandrel in the mandrel mold.

19. A stent formed by the process of claim 17, wherein forming the mandrel includes placing powdered metal in the mandrel mold.

20. A stent formed by the process of claim 16, wherein the stent pattern includes a rounded trough surface.

21. A stent formed by the process of claim 16, wherein filling the stent pattern with the stent material includes depositing the stent material in the stent pattern using chemical vapor deposition.

22. A stent formed by the process of claim 16, wherein filling the stent pattern with the stent material includes filling the stent pattern with a porous stent material.

23. A stent formed by the process of claim 22, further comprising saturating the porous stent material with a fluid.

24. A stent formed by the process of claim 16, wherein the process further comprises forming pores in at least a portion of the stent material.

25. A stent formed by the process of claim 24, further comprising saturating the porous portion of the stent material with a fluid.

26. A stent formed by the process of claim 16, wherein filling the stent pattern with the stent material includes:
providing a first layer of a first stent material in the stent pattern; and
providing a second layer of a second stent material in the stent pattern.

27. A stent formed by the process of claim 26, wherein the second layer includes a radiopaque material.

28. A stent formed by the process of claim 16, wherein filling the stent pattern comprises overfilling the stent pattern.

29. A stent formed by the process of claim 28, further comprising removing an excess portion of the stent material from the mandrel.

30. A stent formed by the process of claim 16, wherein dissolving the mandrel comprises partially dissolving the mandrel and then fully dissolving the mandrel, and wherein the method further comprises changing the configuration of the stent material after the partial dissolution of the mandrel and before the full dissolution of the mandrel.

31. A stent-forming element, comprising:
a mandrel comprising a soluble material, wherein the mandrel includes a surface having a stent pattern defined therein; and
a stent in the stent pattern.

32. A stent-forming element according to claim 31, wherein the mandrel comprises a molded mandrel.

33. A stent-forming element according to claim 32, wherein the mandrel comprises a metal injection molded mandrel.

34. A stent-forming element according to claim 31, wherein the stent pattern includes a rounded trough surface.

35. A stent-forming element according to claim 31, wherein the stent includes a porous material.

36. A stent-forming element according to claim 35, wherein the porous material is saturated with a fluid.

37. A stent-forming element according to claim 31, wherein the stent includes
a first layer of a first stent material; and
a second layer of a second stent material.

38. A stent-forming element according to claim 37, wherein the second layer includes a radiopaque material.

39. A stent-forming element according to claim 31, wherein the stent pattern is overfilled with the stent.

40. A stent-forming element according to claim 31, wherein the mandrel is partially dissolved away from the stent.

41. A mandrel for forming a stent, the mandrel comprising a soluble material having a surface, wherein the surface has a stent pattern formed therein.

42. A mandrel according to claim 41, wherein the mandrel comprises a molded mandrel.

43. A mandrel according to claim 42, wherein the mandrel comprises a metal injection molded mandrel.

44. A mandrel according to claim 41, wherein the stent pattern includes a rounded trough surface.

45. A mandrel according to claim 41, further comprising a stent material in the stent pattern.

46. A mandrel according to claim 45, wherein the stent material includes a porous material.

47. A mandrel according to claim 46, wherein the porous material is saturated with a fluid.

48. A mandrel according to claim 45, wherein the stent material includes:
a first layer of a first stent material; and
a second layer of a second stent material.

49. A mandrel according to claim 48, wherein the second layer includes a radiopaque material.

50. A mandrel according to claim 41, wherein the stent pattern is overfilled with a stent material.

51. A mandrel according to claim 41, wherein the mandrel is partially dissolved away from a stent material in the stent pattern.

52. A method of making a device, comprising:
providing a mandrel having a device pattern;
filling the device pattern with a device material; and
dissolving the mandrel.

53. A method of making a device according to claim 52, further comprising providing a mandrel mold having the device pattern, and wherein providing the mandrel includes forming the mandrel in the mandrel mold.

54. A method of making a device according to claim 53, wherein forming the mandrel in the mandrel mold includes metal injection molding the mandrel in the mandrel mold.

55. A method of making a device according to claim 53, wherein forming the mandrel includes placing powdered metal in the mandrel mold.

56. A method of making a device according to claim 52, wherein the device pattern includes a rounded trough surface.

57. A method of making a device according to claim 52, wherein filling the device pattern with the device material includes depositing the device material in the device pattern using chemical vapor deposition.

58. A method of making a device according to claim 52, wherein filling the device pattern with the device material includes filling the device pattern with a porous device material.

59. A method of making a device according to claim 58, further comprising saturating the porous device material with a fluid.

60. A method of making a device according to claim 52, further comprising forming pores in at least a portion of the device material.

61. A method of making a device according to claim 60, further comprising saturating the porous portion of the device material with a fluid.

62. A method of making a device according to claim 52, wherein filling the device pattern with the device material includes:
providing a first layer of a first device material in the device pattern; and
providing a second layer of a second device material in the device pattern.

63. A method of making a device according to claim 62, wherein the second layer includes a radiopaque material.

64. A method of making a device according to claim 52, wherein filling the device pattern comprises overfilling the device pattern.

65. A method of making a device according to claim 64, further comprising removing an excess portion of the device material from the mandrel.

66. A method of making a device according to claim 52, wherein dissolving the mandrel comprises partially dissolving the mandrel and then fully dissolving the mandrel, and wherein the method further comprises changing the configuration of the device material after the partial dissolution of the mandrel and before the full dissolution of the mandrel.
